(19) United States
(12) Patent Application Publication
(10) Pub. No.: US 2009/0005803 A1
(43) Pub. Date: Jan. 1, 2009

(54) Removable vascular filter and method of filter use

(76) Inventor: Stanley Batiste, Granite Bay, CA (US)

Correspondence Address: WEIDE & MILLER, LTD., 7251 W. LAKE MEAD BLVD., SUITE 530, LAS VEGAS, NV, 89128, US

(21) Appl. No.: 12/215,708

(22) Filed: Jun. 26, 2008

Related U.S. Application Data
(60) Provisional application No. 60/937,506, filed on Jun. 27, 2007.

(51) Int. Cl. A61M 29/00

(52) U.S. Cl. 606/200

(57) ABSTRACT

A vascular filter system and method of removing the same is disclosed. In one embodiment, the filter system comprises a filter housing having a plurality of hollow support struts connected to a support ring, and a filter element having a plurality of primary filter limbs. Securing barbs extend from the distal ends of the primary filter limbs. The primary filter limbs are slidably held within the hollow support struts of the filter housing to form the filter system. The hollow support struts hold the filter element away from a vascular wall to allow only the securing barbs to contact the wall. The filter system may be removed by advancing a snaring catheter and wire to the filter system and drawing the system into the catheter thereby causing the securing barbs to retract into the hollow support struts releasing the system from the vein.

### CROSS-REFERENCE TO RELATED APPLICATIONS

This application claims priority to U.S. Provisional Patent Application No. 60/937,506 filed on Jun. 27, 2007 titled REMOVABLE VASCULAR FILTER AND METHOD OF FILTER USE, and U.S. patent application Ser. No. 12/069,369 filed on Feb. 7, 2008 titled REMOVABLE VASCULAR FILTER AND METHOD OF FILTER PLACEMENT which claims priority to U.S. Provisional Patent Application No. 60/900,378 filed on Feb. 7, 2007 and U.S. Provisional Patent Application No. 60/904,547 filed on Mar. 2, 2007.

### BACKGROUND OF THE INVENTION

1. Field of the Invention

The present invention relates to vascular filters and, in particular to implanted vascular filters which capture thrombi (blood clots) and prevent the thrombi from migrating to other regions of the circulatory system.

2. Related Art

Deep vein thrombosis (DVT) is a common problem and causes significant morbidity and mortality in the United States and throughout the world. DVT is caused when a blood thrombus forms in the deep veins of the legs. These blood thrombi typically occur due to slow or reduced blood flow through the deep veins such as when the patient cannot ambulate or otherwise efficiently circulate their blood. Another cause of inefficient circulation may be due to structural damage to the veins such as general trauma or subsequent to surgical procedures. Additionally, a blood thrombus may form in a deep vein due to a particular medical condition or a propensity for the patient to have a hypercoagubility state. For example, a woman on birth control who smokes has an increased risk of forming blood thrombi and is thus predisposed to DVT.

The result and clinical significance of DVT is when the thrombus breaks free from its location in the deep vein of the leg, the thrombus travels through the circulatory system and may eventually lodge in a location that is adverse to the patient's health. For example, the thrombus may dislodge from a location in the deep vein of the patient's leg and migrate through the heart and come to rest in the patient's lung causing a pulmonary embolism (PE) resulting in restricted circulation and possibly sudden death for the patient.

DVT & PE are currently prevented in several ways including anticoagulation therapy, thrombectomy, thrombolysis and inferior vena cava filter (IVC filter) placement. Anticoagulation therapy utilizes various medications that reduce the patient's propensity for forming blood thrombi. However, this form of therapy has the disadvantage that due to the patient's inability to form blood thrombi (due to the medication), there is an increased risk of excessive bleeding should the patient become injured, sustain surgical complications or develop internal hemorrhaging.

Thrombectomy is a procedure generally performed for treatment of a PE, in which a blood thrombus is extracted from the vein using a surgical procedure or by way of an intravenous catheter and a mechanical suction device. This form of treatment is risky and technically very difficult because the catheter has to be advanced through the vascular system and navigated to a specific location in order to extract the thrombus. Additionally, during a thrombectomy there is an increased risk of causing vascular damage due to the surgical procedure and use of various mechanical devices.

Thrombolysis is a medical technique that is generally performed for treatment of a PE, in which various medicines are infused into the region of the thrombus that subsequently cause the thrombus to dissolve. This form of treatment has the disadvantage potential medication induced bleeding at other sites such as within the brain. For example, if a patient has previously had a tiny non-clinical stroke, the medication used to treat a thrombolysis may cause a previously healed vessel to bleed within the patients head.

IVC filters have been very successful in saving countless lives and are the mainstay of treatment in a population of patients predisposed to deep vein thrombosis (DVT) and pulmonary embolism (PE). IVC filter placement is usually conducted by surgically installing a filter in a large bore vein such as the inferior vena cava located in the patient's upper abdomen (See FIG. 1). The IVC filter is placed using a large bore catheter (Introducer Catheter) for delivery of the filter. There are several existing filters available for patient placement, some are permanent and some are removable for a limited time, after which the removable filter becomes permanent. In the case where a removable filter is utilized, additional complications arise when the filter must be removed. The known removable IVC filter is generally placed for a time period of a several weeks to a few months to prevent internal vascular scaring. However, removal of the current IVC filters is technically challenging and requires large bore access either through the internal jugular vein of the patient's neck or the common femoral vein.

The currently available IVC filters are all limited in their ability to be efficiently and safely removed from the patient after a predetermined time interval. In addition, although the current designs are approved for several weeks or months such prior art designs can be extremely difficult to remove. In addition, prior art designs cause injury to the vascular wall because over even short periods of time, prior art filters become attached to the vascular wall.

Previous attempts to create a filter which is adequately attached to the vascular wall yet will not scar in place have not met with success to date. As a result, there is a need in the art for a removable IVC vascular filter that overcomes the drawbacks of the prior art. The vascular filter and its method of use as described herein enables a physician to place and remove an IVC filter with minimal risk of vascular damage while at the same time increasing the time period by which the filter may be safely removed.

### SUMMARY OF THE INVENTION

Generally, the vascular filter system or assembly comprises a resilient filter housing and a resilient filter element. Both the filter housing and the filter element are resilient in that they are designed to be flexible and fully collapsible. In one embodiment, the filter housing comprises a plurality of hollow support struts connected together. The filter element comprises a plurality of primary filter limbs having securing barbs and a retrieval hook. The filter element is suspended within the filter housing such as by slidably inserting one or more of the primary filter limbs into the hollow support struts of the filter housing. In this embodiment, the securing barbs at the ends of the primary filter limbs extend radially out of the distal ends of the hollow support struts.

In one or more embodiments, the filter system's filter element includes a first element end and a second element end. In these embodiments, at least one secondary limb extends from the first element end to the second element end. The filter element may also include a central connection point located at the first element end. The central connection point of one or more embodiments provides a connection point from which the primary filter limbs, secondary filter limbs, and retrieval hook may extend.

As stated, the primary filter limbs of the filter element are slidably inserted in the hollow support struts of the filter housing. The filter element may be supported by the filter housing in various ways however. In some embodiments, the filter housing's hollow support struts may extend from a support ring. The filter housing may also include retention hooks extending from the support ring and engaging the filter element. In addition, the filter system may be configured such that more than one of the primary filter limbs may be inserted into one hollow support strut. The hollow support struts in one or more embodiments may include at least one intermediate opening allowing a support barb to extend there through.

In one embodiment, the vascular filter system comprises a support ring, a plurality of hollow support struts extending from the support ring, a central connection point adjacent to the support ring, a plurality of primary filter limbs extending from the central connection point and having a distal end and a securing barb extending radially outward there from, and a retrieval hook extending from the central connection point. One or more of the hollow support struts may have at least one of the primary filter limbs slidably inserted therein. The vascular filter system may be formed from resilient material.

In this embodiment, the vascular filter system may have a teardrop shape. In addition, the vascular filter system may further comprise a spacing bend formed into at least one of the securing barbs, at least one intermediate opening on one or more of the hollow support struts, an inwardly angled support flange at a distal end of one or more of the hollow support struts, at least one secondary filter limb extending from the central connection point, or a combination thereof.

A method for implanting a vascular filter assembly comprising is also disclosed herein. In one embodiment, the method comprises accessing a vein and advancing a guiding wire to a predetermined location, advancing a deployment sheath over the guiding wire, removing the guiding wire, advancing a filter assembly within the deployment sheath, advancing a deployment member within the deployment sheath, retracting the deployment sheath, and removing the deployment sheath and deployment member from the vein.

In this embodiment, retracting the deployment sheath releases the filter assembly allowing the filter assembly to expand within the vein and the one or more securing barbs to engage an inner wall of the vein. According to the method, the filter element may comprise a spacing bend formed into at least one of the securing barbs, and the filter assembly may be formed from resilient material.

A method for removing a filter assembly is disclosed herein as well. The filter assembly to be removed may be engaged to a vein by one or more securing barbs and comprise a filter housing having a plurality of hollow support struts and a filter element having a plurality of primary filter limbs, at least one of the primary filter limbs having one of the securing barbs extending there from.

In one embodiment, the method of removal comprises accessing a vein and inserting a snaring catheter having a snaring wire disposed therein, advancing the snaring catheter until the snaring catheter is adjacent to the filter assembly, advancing the snaring wire until the snaring wire engages a retrieval hook of the filter assembly, retracting the retrieval hook to draw the securing barbs of the filter element into the hollow support struts of the filter housing such that filter is disengaged from the vein, and advancing the snaring catheter over the filter housing to substantially contain the filter within the snaring catheter. The snaring catheter and filter assembly contained therein may then be removed from the vein.

The method for removing a filter may further comprise the step of advancing a secondary catheter, larger than the snaring catheter, along the snaring catheter until the filter assembly is contained within the secondary catheter. According to the method, the filter element may comprise a spacing bend formed into at least one of the securing barbs, and the filter assembly may be formed from resilient material.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 illustrates a typical filter placement within the Inferior Vena Cava.

FIG. 2 illustrates two types of existing inferior vena cava filters.

FIGS. 3A through 3C illustrate exemplary embodiments for an inferior vena cava filter including a filter element and a filter housing individually and combined in both an expanded and collapsed state.

FIG. 4 is an enlarged detail area that illustrates a support ring and hollow support struts of the filter housing.

FIG. 5 is an enlarged detail area that illustrates the filter element assembled into the filter housing.

FIG. 6A is an enlarged detail area that illustrates a securing barb of the filter element's primary filter limb.

FIGS. 6B through 6E illustrate alternate embodiments of the inferior vena cava filter.

FIG. 7 illustrates the collapsed filter housing and filter element assembly as contained within a deployment sheath.

FIGS. 8A through 8G illustrate the deployment of the filter housing and filter element assembly.

FIGS. 9A through 9H illustrate the removal of the inferior vena cava filter.

### DETAILED DESCRIPTION OF THE PREFERRED EMBODIMENTS

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

One of the primary concerns regarding deep vein thrombosis (DVT) is that should the thrombus (blood clot) dislodge from the origination location, the thrombus may travel to another region of the circulatory system and cause injury and or death to the subject. For example, if a DVT dislodges it may migrate through the heart and eventually re-lodge in the lung of the subject thus causing a Pulmonary Embolism which prevents adequate circulation and can cause sudden death of the subject. By placing an intravenous filter in the Inferior Vena Cava (IVC), the thrombus may be captured and prevented from migrating to vulnerable regions of the circulatory system. The filter may be placed in other veins or at other locations such that the filter is positioned to capture a thrombus prior causing damage or medical complications to the patient.

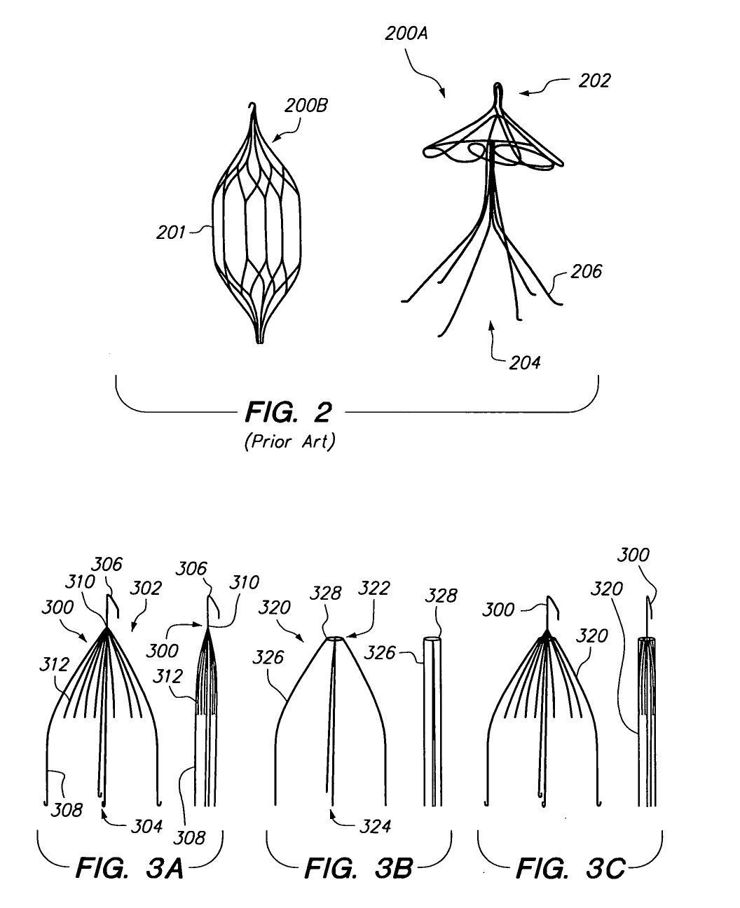

A major design problem with existing IVC filters is that numerous prior art filter designs have some component that opposes the wall of the vessel. This is either by “side struts” 200 (See FIG. 2) or by “limbs” 206 that radiate outward. These struts or limbs prevent filter migration within the vein to undesirable locations such as the heart.

One problem regarding current filter removal is caused by the struts or limbs embedding and adhering to the vascular wall. The embedding and adherence is effectuated by the formation of scar tissue between the filter components (side-struts or limbs) and the tissue of the vascular wall. In order for the IVC filter to have enough grip within the vessel wall and prevent filter displacement, a significant amount of surface area of the filter must directly oppose in friction fit with the vascular wall. Over time, scar tissue will envelope and securely attach to the filter components resulting in a filter that cannot be adequately removed without a substantial risk of vascular damage. Scarring in place, embedding and adherence are the reasons existing IVC filter designs are approved for in-body placement for a limited time. This avoids having a physician removing a filter that has become permanently embedded within the vascular wall thereby causing damage to the vascular system.

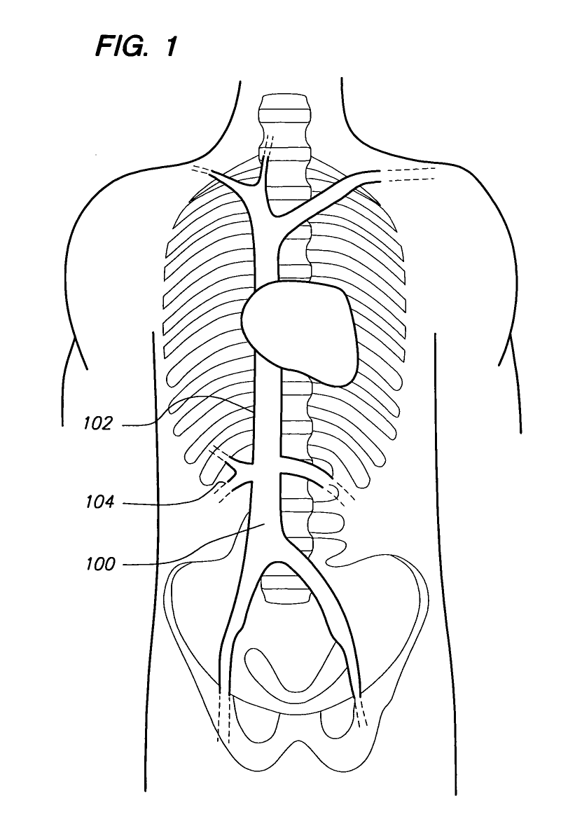

Referring now to the drawings, FIG. 1 illustrates the typical location 100 for surgically implanting an IVC filter using a large bore vein such as the IVC 102 located in the patient's upper abdomen. The IVC filter is typically deployed within the large bore vein using a large bore catheter and traditional access through a larger vein such as the patient's common femoral vein, the veins of the upper arm or the internal jugular vein. An IVC filter is generally placed within the IVC 102 below the renal veins 104 as illustrated in FIG. 1.

FIG. 2 illustrates two types of existing inferior vena cava vascular filters that are surgically implanted into a patient. The inferior vena cava filter (IVC filter) 200A, 200B is commonly deployed using a large bore catheter and access to a large bore vein such as the inferior vena cava. The typical IVC filter 200A has a first end 202 and a second end 204 where the second end comprises a plurality of individual wire components 206 or limbs that are in contact with the vascular walls during deployment. In another version of the typical IVC filter 200B the filter is generally cylindrical in shape and has a plurality of side-strut edges 201 that engage the inner vascular walls during deployment.

FIGS. 3A through 3C illustrate a filter element 300, a filter housing 320 and the assembly of both an expanded and collapsed state. As shown in FIG. 3A, a filter element 300 comprises a first element end 302 and a second element end 304 and is generally tear-drop in shape. The filter element 300 has a retrieval hook 306 that is integrally formed from the first element end 302 and extends outwardly there from. The retrieval hook 306 facilitates the removal process by providing a centrally located grasping point on the filter element 300 by which a snaring loop may be attached and the filter element subsequently drawn into a removal catheter. A detailed disclosure of the removal process is provided below.

The filter element 300 comprises a plurality of primary filter limbs 308 that extend or curve away from a central connection point 310 on the first element end 302 towards the second element end 304. The filter element 300 has a plurality of secondary filter limbs 312 that partially extend from the central connection point 310 towards the second element end 304. The filter element 300 is sized for insertion within the filter housing 320. In one embodiment, there are 4 primary filter limbs 308 and 8 secondary filter limbs 312 that extend from the center connection point 310; however, it is contemplated that more or less limbs or any combination of primary or secondary limbs may be used. In one embodiment of filter element 300, there are only a few primary filter limbs 308 provided for insertion of the filter element 300 to the filter housing 320. By reducing the number of primary filter limbs 308, the IVC vascular filter assembly can be fabricated to fit within a smaller catheter because there are less limbs directly attached to the filter housing 320 and correspondingly occupy less volume.

The filter element 300 further comprises a plurality of secondary filter limbs 312 which provide filtering means for the regions between the primary filter limbs 308. As a result, the secondary filter limbs 312 are not connected or attached to the filter housing 320 and generally extend from the first element end 302 towards the second element end 304. The secondary filter limbs 312 may be either curved, straight or a combination of geometric transformations (such as spiral, vortex, etc) extending from the first element end 302. An important aspect of the secondary filter limbs 312 is that they are not attached to or contained within a hollow support strut of the filter housing 320 but rather occupy the volumetric region between the primary filter limbs 308 and provide a means for filtering/capturing thrombi flowing through the IVC filter.

The filter element 300 is preferably fabricated from a suitable material such as titanium, Nitinol® to name a few or any plastic or synthetic material. The wire may be similar to known wires commonly used in the medical industry and may range in diameter from 0.015-0.035 of an inch. Additionally, the filter element 300 may be coated with a compound that prevents clot formation such as a Heparin anticoagulation coating. The filter element may comprise a mesh form or may be constructed of metal, plastic or a combination thereof or any other material suite for medical device implementation.

The filter housing 320, shown in FIG. 3B, comprises a first housing end 322 and a second housing end 324 and the housing is generally tear-drop or ogive in shape, a plurality of longitudinal hollow support struts 326 that are configured and arranged to coincide with the primary filter limbs 308 of the filter element 300. The filter housing 320 has a support ring 328 located at the first housing end and is used to provide structural support for the hollow support struts 326. The hollow support struts 326 generally extend from the support ring 328 towards the second housing end 324. The hollow support struts 326 suspend, support and space the filter limbs away from the inner vascular walls. By spacing the filter limbs away from the inner vascular wall, the filter element 300 will not or are less likely to significantly contact, scar-in or otherwise adhere to the vascular wall. An important aspect of the filter housing 320 is to provide a member that releasably retains a filter element while at the same time constraining the filter limbs such that the limbs will not significantly or not at all contact the vascular wall. In one embodiment, the filter housing maybe coated with a compound that prevents clot formation such as a Heparin anticoagulation coating. As a result the anticoagulation coating inhibits or prevents the growth of scar tissue that might adhere to the filter housing structure.

As shown in FIG. 3C, the filter element 300 and filter housing 320 are assembled into a single filtration device of the present invention. In one embodiment both the filter housing 320 and filter element 300 are designed to be flexible, resilient and fully collapsible so that they may be advanced, as a single assembly, into a vascular region using a catheter sheath. Note that the term resilient, in one or more embodiments, generally means that the filter housing 300 and the filter element 320 are flexible and may fully or partially collapse and substantially or completely recover their original shape. The steps involved in deployment and removal are discussed in greater detail below. The filter housing 320 and filter element 300 are contemplated to be fabricated from a material suitable for implantation within a biological subject. Some examples of suitable materials are titanium, polycarbonate, polypropylene or other hypoallergenic materials that provide adequate spring tension, form-factor/shape memory and compatibility with living tissue.

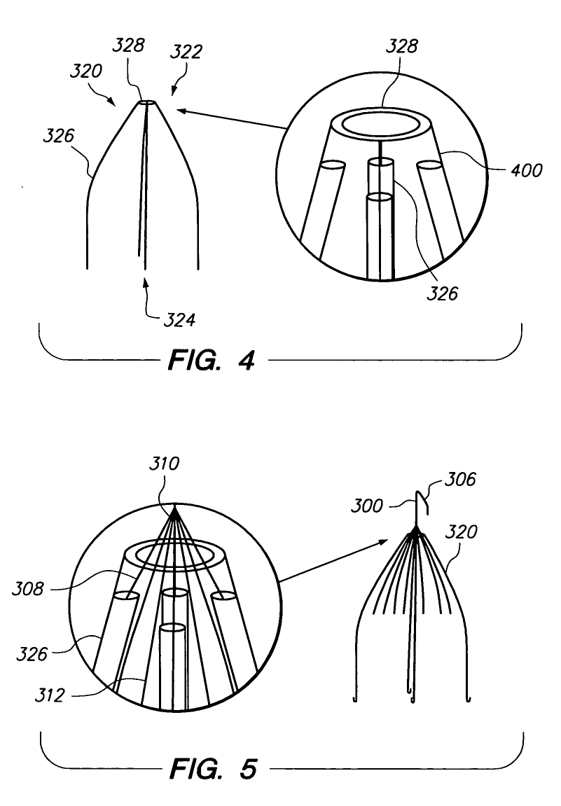

Reference is now made to FIGS. 4 and 5 which provide an enlarged detail illustration of the first housing end 322 and support ring 328. In FIG. 4, the support ring 328 provides the structural support and fastening means for the longitudinal hollow support struts 326. The geometric spacing and arrangement of the hollow support struts 326 may be connected to the support ring 328 by a thin filament 400 which is integrally formed from the support ring 328. FIG. 5 illustrates an enlarged view of the filter housing 320 with the filter element 300 assembled therein. As shown, the primary filter limbs 308 are slideably inserted into the longitudinal hollow support struts 326. The primary filter limbs 308 are thus able to translate or slide within the hollow support struts 326. It is contemplated that the filter housing 320 may provide hollow support struts 326 for each primary filter limb 308. Additionally, the first element end 302 and central connection point 310 of the filter element 300 are located in the center of the support ring 328 and the retrieval hook 306 passes through the center of the support ring 328.

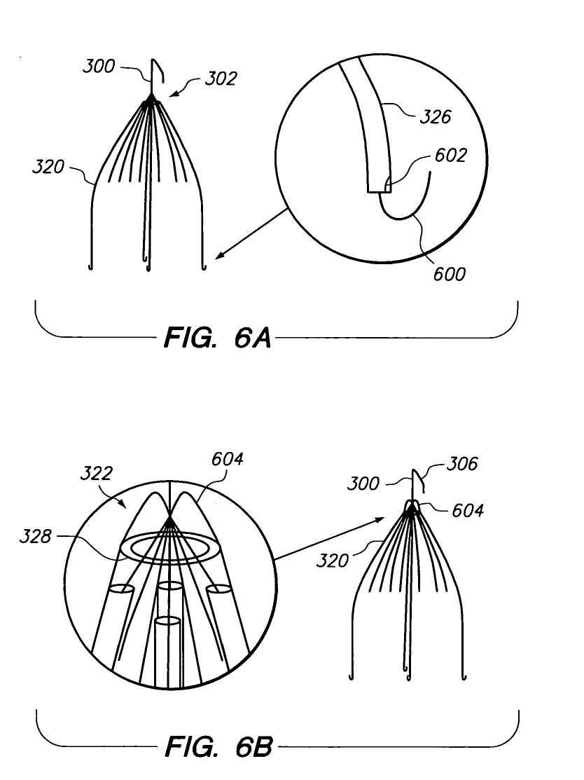

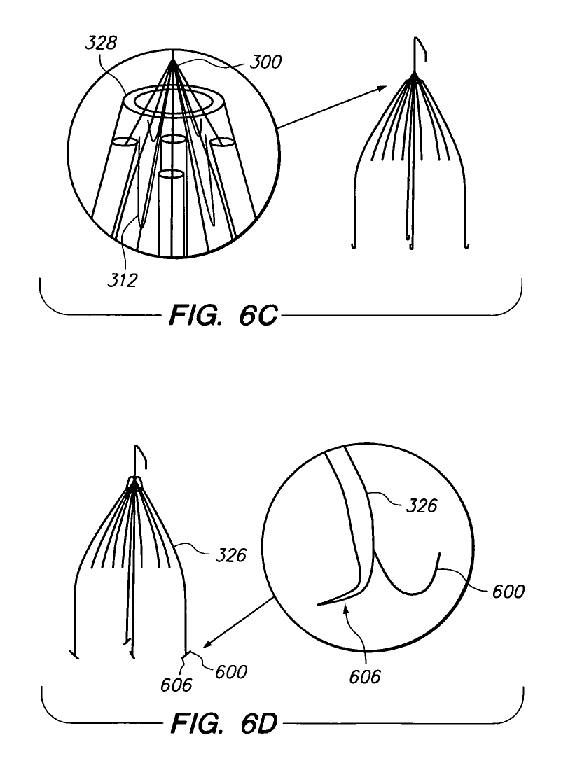

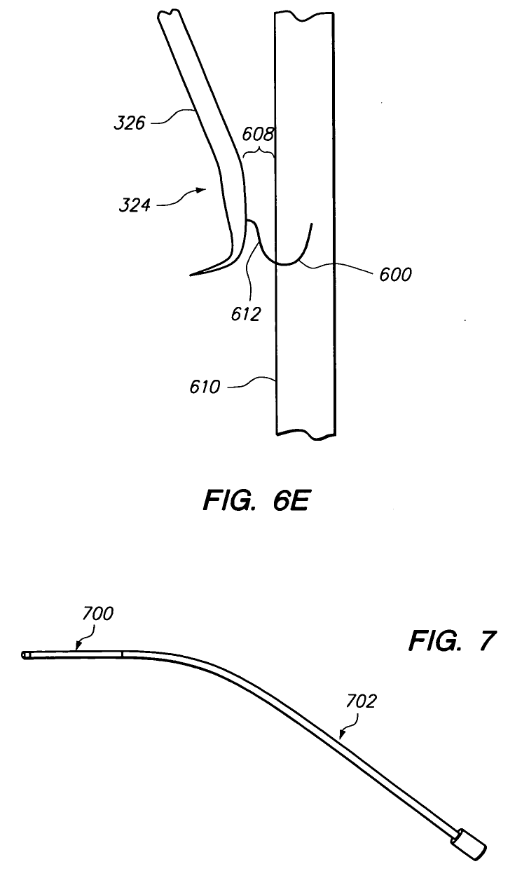

Reference is now made to FIG. 6A, which is an enlarged detail view illustrating a securing barb 600 of the filter element 300. The securing barb 600 extends radially outward from each distal end of the primary filter limbs and curves back towards the first element end 302.

It is contemplated that each end of the primary filter limbs is fitted with the securing barbs 600 such that the barbs resist and prevent movement of the filter assembly with respect to the inner surface of the vascular wall while in a deployed state. The distal end of the longitudinal hollow support struts 326 may be configured to have recess 602 formed thereon that facilitates retraction of the filter element 300 with respect to the filter housing 320. The recess 602 functions to guide the primary filter limb 308 and securing barb 600 into the hollow support strut 326 and provides an opposing support surface that engages the vascular wall during the retrieval process.

In another embodiment, there may be more than one securing barb 600 protruding from the hollow support strut 326. For example, the hollow support strut 326 may have one or more intermediate openings along the strut's length. The intermediate opening may have another primary filter limb 308 extending there through and having a securing barb extending form the limb's end. It is contemplated that that more than one primary filter limb 308 may be inserted into a single hollow support strut 326. The additional filter limbs 308 may have various lengths and exit the hollow support strut 326 at a location other then the distal end of the hollow support strut 326. For example, the hollow support strut may have an intermediate opening midway along the strut's length. Through this opening one of the shorter primary filter limbs may extend, where the limb has a securing barb configured from the end of the limb. As a result, this embodiment may have additional securing barbs that engage the vascular wall at different locations and will provide enhanced engagement with the vascular wall.

The securing barbs 600 penetrate and engage the vascular wall such that the filter element/housing 300,320 will remain in place and cannot move and/or translate within the vein during deployment. It is further contemplated that the securing barbs 600 are integrally formed with the primary filtration limbs 308 of the filter element 300. The securing barb 600 may have other geometric shapes such as an angled barb (as opposed to a curved barb), compound curve-shape or other protrusion that extends away from the filter element/housing assembly and embeds into the vascular wall.

One alternate embodiment is shown in FIG. 6B, which illustrates a modified first housing end 322. In this embodiment, the support ring 328 has one or more retention hooks 604 that extend from the first housing end 322 towards the retrieval hook 306 of the filter element 300. The retention hooks 604 are shaped and configured to engage the filter element 300 to prevent complete separation of the filter element 300 from the filter housing 320. It is contemplated that the retention hooks 604 may be other geometric configurations but provide the basic functionality of retaining the filter element within the filter housing when the securing barbs 600 are retracted into the hollow support struts 326. In operation, the retention hooks 604 provide a safety mechanism that ensures that both the filter element and housing are removed as a single assembly.

In another embodiment illustrated in FIG. 6C, the secondary filter limbs 312 may be configured to latch/hook onto the support ring 328 such that the filter element 300 is retained within filter housing 320 when the securing barbs 600 are retracted into the hollow support struts 326. It is further contemplated that the one or more of the secondary filter limbs 312 may form a loop (not shown) that encircles the support ring 328 such that the support ring 328 passes through the loop. The length of the loop may be configured or sized to permit a predefined displacement between the filter element 300 and the filter housing 320. For example, the loop may be sized so that the loop engages the support ring when the securing barbs are completely retracted into the hollow support struts 326. The retention loop provides another configuration for a safety mechanism that ensures that both the filter element and housing are removed as a single assembly and do not inadvertently separate during the filter removal procedure.

Another embodiment is illustrated in FIGS. 6D and 6E, which shows a modified distal end of the hollow support strut 326 that contains an inwardly angled support flange 606. The support flange 606 is configured to avoid contact with the interior vascular wall during the deployed condition. Due to the inward orientation of the support flange 606 during deployment the distal ends of the hollow support struts 326 are independent and spaced away from the vascular wall (see 608 of FIG. 6D). The spacing 608 prevents/prohibits attachment of the distal end of the hollow support strut 326 to the vascular wall 610 (i.e., scarring in). It is contemplated that the support flange 606 may have various geometric configurations such as a curved surface, arcuate surface, spherical or other geometric shape. Additionally, the support flange 604 may be shaped (in width, depth and length) such that it contours the inner portion of the vascular wall 610. Upon filter removal, when the filter element 300 is retracted with respect to the filter housing 320 and the securing barbs 600 are drawn from the vascular wall 610, the support flange 606 provides opposing supportive force to the interior of the vessel walls. Since the vascular wall is likely to deflect inward and follow the securing barb 600 during removal, the support flange 606 provides a temporary opposing structure that supports the vascular wall and permits the securing barb 600 to be removed from the vessel wall while limiting the deflection of the vessel wall.

Additionally, as shown in FIG. 6E, the configuration of the securing barb 600 may be modified to provide structure that spaces the filter housing 320 second end 324 from the vascular wall 610. It is contemplated that the securing barb 600 may have a spacing bend 612. The spacing bend 612 provides additional clearance or spacing 608 between the distal end of the hollow support strut 326 and the vascular wall 610 during deployment.

Turning now to FIG. 7 which illustrates an assembled IVC filter assembly 700 comprising a filter element and filter housing as previously illustrated in FIG. 3. The assembled IVC filter 700 is illustrated in a collapsed state (as shown in FIG. 3C) and contained within a deployment sheath 702. In FIG. 3C, the IVC filter assembly 700 is collapsed and occupies a substantially smaller volumetric region such that the filter may be placed within a deployment sheath 702. The deployment sheath 702 and IVC filter 700 is generally preloaded by the manufacturer.

The following disclosure is directed to one implementation for deployment and removal of the IVC filter described herein. Although the deployment is being illustrated in one specific orientation, other orientations for filter deployment and removal are possible and known to those of ordinary skill in the field of intravascular surgical procedures.

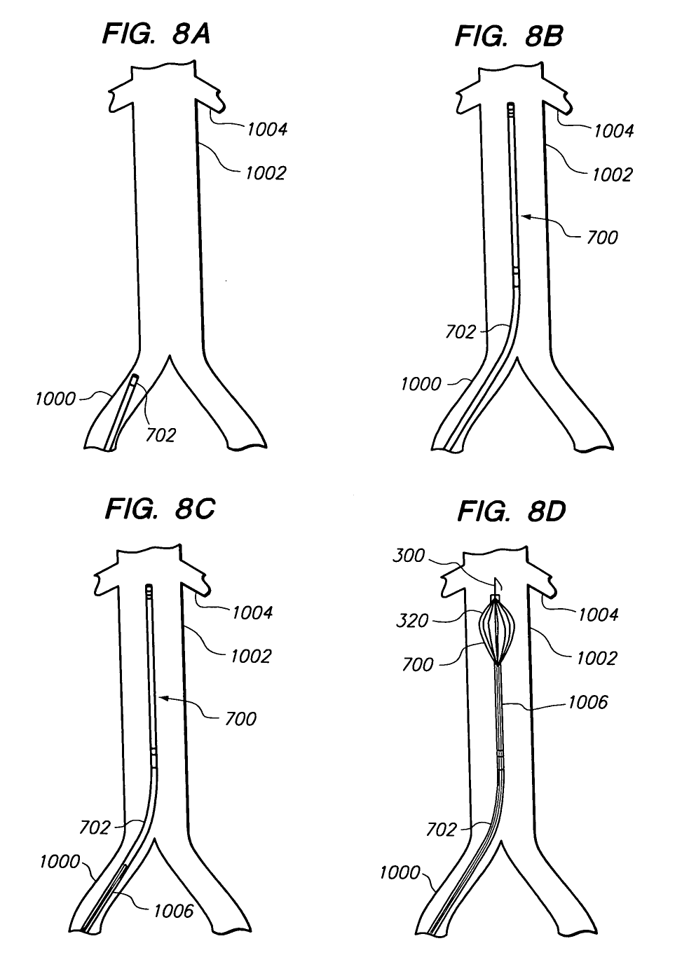

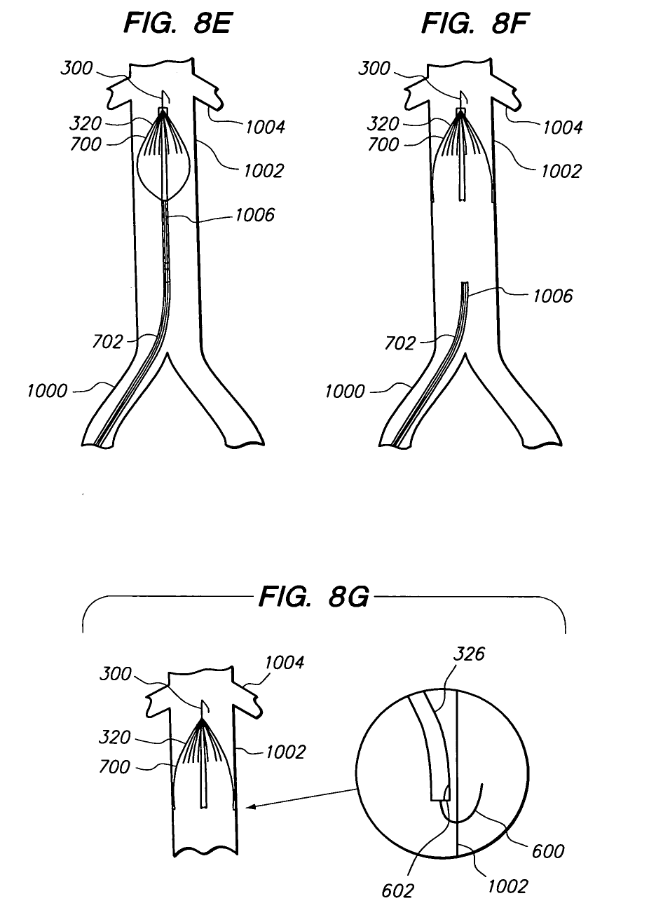

Reference is now made to FIGS. 8A through 8G individually and in combination for illustrating the deployment of the IVC filter assembly 700. The IVC filter assembly 700 is deployed into the inferior vena cava in a similar fashion as most of the current IVC filters. Access is performed using standard techniques into a patient's vein. The veins that are commonly used include the large veins of the groin, such as the common femoral vein 1000, the larger veins of the upper arm or the large vein in the neck—the internal jugular vein. Once access is obtained, a guiding wire is advanced into the inferior vena cava. Over this wire (not shown for clarity) the deployment sheath 702, which is preloaded with the IVC filter assembly 700, is advanced in to the inferior vena cava 1002, see FIG. 8A. Current practice uses contrast (radiographic dye) injected to provide visual navigation and mapping of the inferior vena cava during the procedure.

As shown in FIG. 8B, the deployment sheath 702 is then advanced to the appropriate position within the inferior vena cava 1002 which is generally below the inflow from the renal veins 1004. The guiding wire may then be withdrawn or removed from the patient. Next, in FIG. 8C, a deployment member, push rod or “pusher” 1006 or is then advanced within the deployment sheath 702 to the base of the collapsed IVC filter assembly 700. The IVC filter assembly 700 is then slowly deployed by holding the pusher 1006 in a fixed position and pulling the outer deployment sheath 702 back, see FIG. 8D. This technique then slowly exposes the collapsed filter while maintaining the filter's position relative to the inferior vena cava 1002. The prior form-factor/shape memory and internal tension of the IVC filter assembly 700 causes it to self-expand and “open” within the inferior vena cava 1002, see FIG. 8E.

It is noted that the deployment sheath 702 may be advanced into the inferior vena cava 1002 without having the IVC filter assembly 700 preloaded therein in some embodiments. In these embodiments, the IVC filter assembly 700 may be advanced within the deployment sheath 702 and into the inferior vena cava 1002 after the guiding wire has been removed. This may be accomplished by pushing the IVC filter assembly 700 within the deployment sheath 702 with a deployment member 1006. Of course, other ways of advancing the IVC filter assembly 700 within the deployment sheath 702 may be used. The IVC filter assembly 700 may then be deployed as described above.

As the IVC filter 700 expands, the filter housing 320 meets the inner wall on the inferior vena cava and the securing barbs 600 slightly penetrate and engage the inner wall, see FIG. 8F through 8G. As illustrated, the filter housing 320 and the filter element expand simultaneously to complete the deployment process. Upon complete self-expansion of the IVC filter assembly 700, the deployment sheath and the pusher are withdrawn from the insertion site and the patient's vascular system, see FIG. 8G.

It is further contemplated that the deployment of the IVC filter assembly may be performed in other vascular regions to prevent thrombus migration. The removable filter disclosed herein may be deployed within other regions of a patient's body as required by the specific medical requirements or case stratagem.

The need to remove a filter arises when a patient is no longer at risk for thrombus formation and the possibility of thrombus migration and pulmonary embolism has subsided. There are complications that can occur when a filter is left in place such as scarring of the inferior vena cava and possible metal fatigue/fracture of the filter. In addition, blood flow is hindered or restricted when the filter remains in place. Correspondingly, it is desirable to remove filters when they are no longer necessary for the patient's health. However, the currently available IVC filters typically remain with the patient for life because there is a small time period in which the filter can be safely removed; outside of this time period there is substantial risk of vascular damage to the patient if filter removal is performed. The present invention provides an IVC filter that can remain deployed within a patient for a significant time period while at the same time is removable throughout this period.

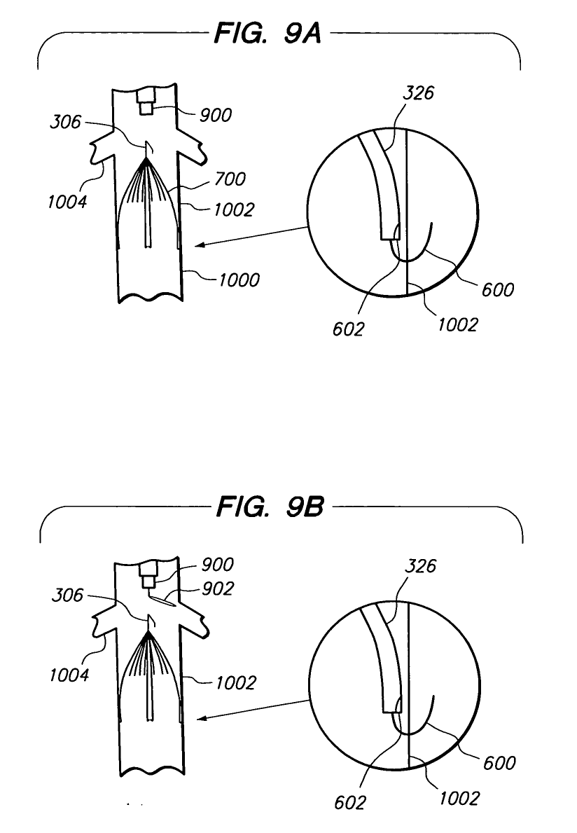

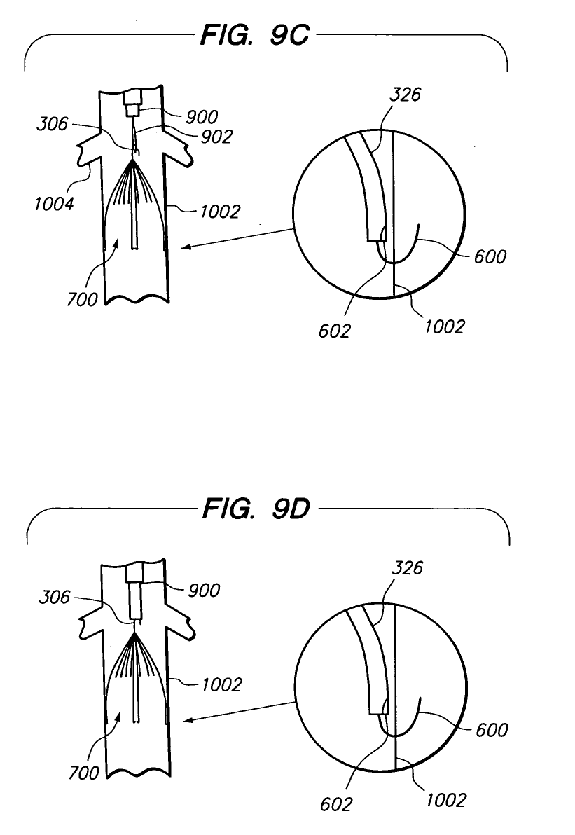

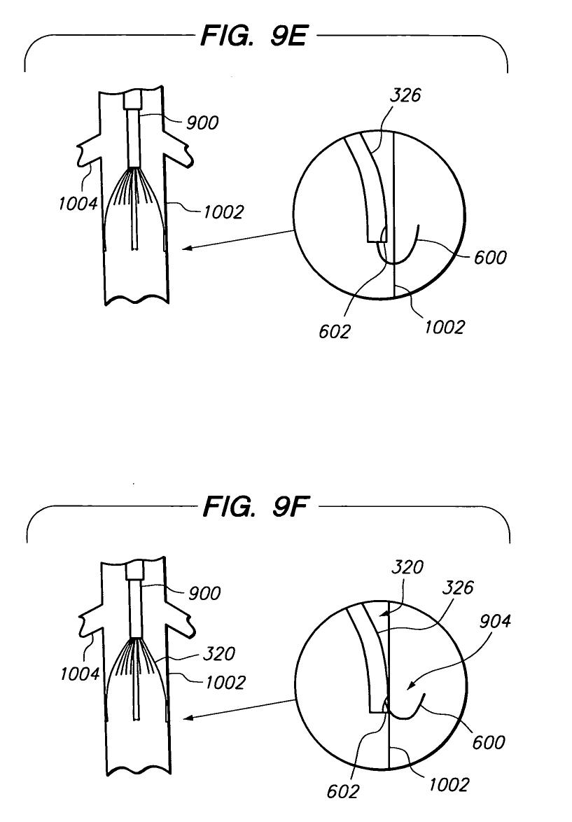

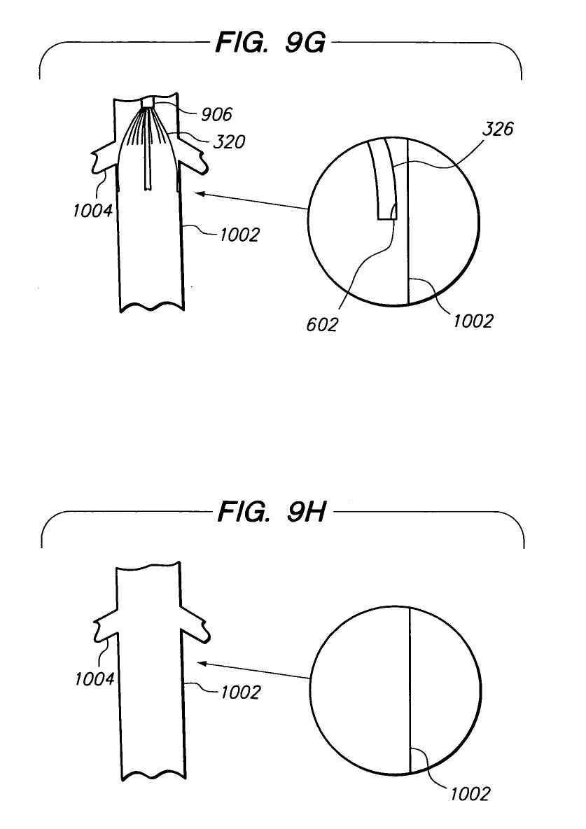

Reference is now made to FIGS. 9A through 9H individually and in combination for illustrating the removal of the IVC filter assembly 700. In FIG. 9A, a snaring catheter 900 having a snare wire therein is advanced to the location of the IVC filter assembly 700 through access performed using standard techniques into a patient's vein. The veins that are commonly used include the large veins of the groin, such as the common femoral vein, the larger veins of the upper arm or the large vein in the neck—the internal jugular vein. The snaring catheter 900 is advanced until within close proximate location with the retrieval hook 306 of the filter element 300 (See FIG. 3A). Once the snaring catheter 900 is in place, the snare wire 902 is advanced through and beyond the end of the snaring catheter 900 as shown in FIG. 9B. The snaring wire 902 is then advanced and manipulated until the snaring wire engages the retrieval hook 306, see FIG. 9B through 9C.

Next, the snaring catheter 900 is advanced along the snaring wire 902 until the catheter is proximate to the retrieval hook 306 as illustrated in FIGS. 9D and 9E. Once the snaring catheter 900 contacts the retrieval hook 306, the snaring wire 902 is retracted through the catheter and the filter element 300 is drawn into the snaring catheter, see FIG. 9E. As the filter element 300 is drawn into the snaring catheter 900, the filter limbs (both primary and secondary) will deflect and slide through support ring 328. The securing barbs 600 deflect and retract into the hollow support struts 326 of the filter housing 320 as illustrated in FIG. 9F. The distal ends of the hollow support struts 326 engage the vascular wall at a location 904 and support the vascular wall to prevent tearing or structural damage to the vascular wall during the retraction process. During the retraction process, the securing barbs 600 are pulled directly out of the inferior vena cava in a substantially perpendicular direction with respect to the vascular wall. The perpendicular direction is facilitated by the recess 602 formed within the distal end of the hollow support strut 326 which cause the securing barbs 600 to pull straight out of the inferior vena cava and thus prevent vascular damage.

In practice, the snaring catheter 900 is slightly advanced in unison while the snaring wire 902 is retracted. The combination of advancing the snaring catheter 900 while retracting the snaring wire 902 is considered a standard snaring technique in the intravascular medical field. In FIG. 9F, the filter element 300 is further drawn into the snaring catheter 900 as the filter housing 320 remains in place. The filter element 300 is continually drawn into the snaring catheter 900 by retracting the snaring wire 902 until the securing barbs 600 detach from the vascular wall of the inferior vena cava, as shown in FIG. 9G. Once the securing barbs 600 are detached, a secondary larger catheter 906 is advanced along catheter 900 and over the filter housing 320. The filter housing 320 then begins to collapse and enter the secondary catheter 906. Once the IVC filter assembly 700 is contained within the larger catheter 906, the catheter with the IVC filter contained therein is removed from the patient and the removal procedure is complete, see FIG. 9H. Conversely, the removal procedure may be performed without the larger catheter 906 such as by advancing the snaring catheter 900 over both the filter element 300 and filter housing 320. In this case, the snaring catheter 900 would need to be adequately sized to contain both the filter element 300 and filter housing 320.

The IVC vascular filter disclosed herein has several advantages over known IVC filters. First, the new vascular filter allows long-term filter removal. In contrast, existing vascular filters are only removable within a limited shorter time interval that may not be adequate for a specific patient's condition. As a result, if the patient requires vascular filtration for a time period that exceeds the removal time interval of current IVC filters, the filter becomes permanently adhered to the patient's vessel and patent will have the filter for life, or the filter must be removed and replaced at a different location thereby necessitating multiple medical procedures.

Secondly, the new IVC vascular filter is completely removable and may be configured to leave no filter structure behind in the patient. Unlike exiting removable filters, which typically leave at least one portion of the filter in the patent after filter retrieval the present filter design is completely removed and leaves no physical structure within the patent.

Thirdly, the new IVC vascular filter provides vascular support during the retrieval procedure. By design, the filter housing provides temporary support to the vascular wall during removal. This temporary support prevents possible vascular damage that may otherwise occur during the retrieval process.

Finally, another advantage of the new IVC vascular filter is reduced fatigue in the filter element. The filter element used in the present invention is contained in the filter housing in a reduced stress/strain environment due to the suspended state configuration. The suspended state configuration is obtained by the use of the filter holding members (hollow support struts) which permit the filter element to float within the filter housing while at the same time being physically constrained within the filter housing. As a result, while the filter housing encapsulates the filter element, the filter housing becomes the stress/strain load path for vascular contractions which in turn removes these forces which would typically be applied to the filter element.

While various embodiments of the invention have been described, it will be apparent to those of ordinary skill in the art that many more embodiments and implementations are possible that are within the scope of this invention. In addition, the various features, elements, and embodiments described herein may be claimed or combined in any configuration or arrangement.

What is claimed is:

1. A vascular filter system comprising:
a resilient filter housing comprising a plurality of hollow support struts having a proximal end and a distal end, the hollow support struts connected at their proximal ends;
a resilient filter element, the filter element comprising a plurality of primary filter limbs and a retrieval hook, the filter element suspended within the filter housing by one or more of the primary filter limbs being slidably inserted into one or more of the hollow support struts, wherein at least one of the primary filter limbs has a securing barb that radially extends out of the distal end of the hollow support strut.

2. The vascular filter system of claim 1, wherein the filter element further comprises at least one secondary filter limb extending from a first element end to a second element end of the filter element.

3. The vascular filter system of claim 2, wherein the filter element further comprises a central connection point at the first element end, the primary filter limbs, secondary filter limbs, and retrieval hook extending there from.

4. The vascular filter system of claim 1, wherein the filter housing further comprises a support ring, the hollow support struts extending from the support ring.

5. The vascular filter system of claim 4, wherein the filter housing further comprises retention hooks extending from the support ring and engaging the filter element.

6. The vascular filter system of claim 4, wherein the secondary filter limbs are configured to hook onto the support ring when the vascular filter system is removed from a vein.

7. A vascular filter system comprising:
a support ring;
a plurality of hollow support struts extending from the support ring;
a central connection point;
a plurality of primary filter limbs extending from the central connection point, at least one of the plurality of primary filter limbs having a distal end and a securing barb at the distal end, the securing barb extending radially outward; and
a retrieval hook extending from the central connection point;
wherein at least one of the plurality of hollow support struts has at least one of the primary filter limbs slidably inserted therein; and
wherein the vascular filter system is formed from resilient material.

8. The vascular filter system of claim 7, wherein the vascular filter system has a teardrop shape.

9. The vascular filter system of claim 7, wherein the securing barb comprises a spacing bend.

10. The vascular filter system of claim 7 further comprising at least one intermediate opening on one or more of the hollow support struts.

11. The vascular filter system of claim 7 further comprising a support flange at a distal end of one or more of the hollow support struts, the support flange being inwardly angled.

12. The vascular filter system of claim 7 further comprising at least one secondary filter limb extending from the central connection point.

13. A method for implanting a vascular filter assembly comprising:
accessing a vein and advancing a guiding wire;
advancing a deployment sheath over the guiding wire;
removing the guiding wire from the deployment sheath;
advancing a filter assembly within the deployment sheath, the filter assembly comprising a filter housing having a plurality of hollow support struts and a filter element having a plurality of primary filter limbs, at least one of the primary filter limbs having one or more securing barbs extending therefrom;
advancing a deployment member within the deployment sheath;
retracting the deployment sheath, wherein retracting the deployment sheath releases the filter assembly allowing the filter assembly to expand within the vein and the one or more securing barbs to engage an inner wall of the vein; and
removing the deployment sheath and deployment member from the vein.

14. The method of claim 13, wherein the filter element further comprises a spacing bend formed into at least one of the securing barbs.

15. The method of claim 13, wherein the filter element further comprises one or more secondary filter limbs.

16. The method of claim 13, wherein the filter assembly is formed from resilient material.

17. A method for removing a vascular filter assembly comprising:
accessing a vein and inserting a snaring catheter, the snaring catheter having a snaring wire disposed therein;
advancing the snaring catheter until the snaring catheter is adjacent to the filter assembly, the filter assembly engaged to a vein by one or more securing barbs and comprising a filter housing having a plurality of hollow support struts and a filter element having a plurality of primary filter limbs, at least one of the primary filter limbs having one of the securing barbs extending there from;
advancing the snaring wire until the snaring wire engages a retrieval hook of the filter assembly;
retracting the retrieval hook, wherein the retraction of the retrieval hook draws the securing barbs of the filter element into the hollow support struts of the filter housing such that filter assembly is disengaged from the vein;
advancing the snaring catheter over the filter assembly, wherein the filter assembly is substantially contained within the snaring catheter; and
removing the snaring catheter and filter assembly contained therein from the vein.

18. The method of claim 17 further comprising the step of advancing a secondary catheter along the snaring catheter until the filter assembly is contained within the secondary catheter, the secondary catheter being larger than the snaring catheter.

19. The method of claim 17, wherein the filter element further comprises a spacing bend formed into at least one of the securing barbs.

20. The method of claim 17, wherein the filter element further comprises one or more secondary filter limbs.

21. The method of claim 17, wherein the filter assembly is formed from resilient material.
